(19) United States
(12) Patent Application Publication
(10) Pub. No.: US 2008/0292663 A1
(43) Pub. Date: Nov. 27, 2008

(54) ADJUVANT COMPOSITIONS AND METHODS FOR DELIVERING VACCINES

(76) Inventor: Jay D. GERBER, Lincoln, NE (US)

Correspondence Address: STITES & HARBISON PLLC, 1199 NORTH FAIRFAX STREET, SUITE 900, ALEXANDRIA, VA, 22314, US

(21) Appl. No.: 12/125,577

(22) Filed: May 22, 2008

Related U.S. Application Data
(60) Provisional application No. 60/939,458, filed on May 22, 2007.

(51) Int. Cl. A61K 39/00; A61K 47/30; A61P 31/00

(52) U.S. Cl. 424/280.1; 514/772.6

(57) ABSTRACT

An adjuvant composition is provided that is comprised of a calcium compound, lecithin, and an acrylic polymer. Pharmaceutical compositions are provided which include an antigen and the adjuvant. Methods are provided for stimulating an immune response in a human or animal subject by administering a composition comprising an antigen and the adjuvant composition to human or animal subjects.

### RELATED APPLICATIONS

This application claims priority from U.S. Provisional Application Ser. No. 60/939,458 filed May 22, 2007, which is incorporated herein by this reference.

### FIELD OF THE INVENTION

The present invention relates to adjuvant compositions and methods for delivering antigens.

### BACKGROUND OF THE INVENTION

The ability of a vaccine antigen to elicit a sufficient immune response in a human or animal subject often depends on the particular adjuvant employed in the vaccine. Adjuvants are generally compounds that, when administered with an antigen (either mixed with, or given prior to the administration of the antigen), enhance and/or modify the immune response to that particular antigen. In other words, the adjuvant used can either reduce the immune response to a particular antigen or enhance the development of a desired immune response.

The use of adjuvants in combination with antigens is not a new or unique method of enhancing an immune response in humans and animals. Chemically, however, the adjuvants in use today are a highly heterogeneous group of compounds that vary in terms of their ability to affect the immune system of a given animal or human and vary in the seriousness of the adverse effects that are observed after administration of a vaccine.

Examples of prior art adjuvants include the following:

U.S. Pat. No. 4,917,892, Speaker et al., issued Apr. 17, 1990, describes a topical delivery system comprising a viscous carrier containing a dissolved or dispersed active agent and active agent microencapsulated within a semi permeable anisotropic salt film which is the emulsion reaction product of a) a partially lipophilic, partially hydrophilic, polyfunctional Lewis acid or salt thereof in aqueous medium, such as carboxymethylcellulose, an alkali metal salt of polyacrylic acid or cross linked polyacrylic acid/polyoxyethylene, with b) a Lewis base or salt thereof in a water-immiscible, slightly polar organic solvent for the base, such as benzalkonium chloride, and piperidine.

U.S. Pat. No. 5,132,117, Speaker et al., issued Jul. 21, 1992, discloses a microcapsule with an aqueous core, capsular, ionic stabilized anisotropic Lewis salt membrane formed from the interfacial reaction product of an emulsion of an aqueous solution of a water-soluble, hydrophilic polymeric Lewis acid or salt thereof with a non-aqueous solution of a lipophilic Lewis base or salt thereof. The Lewis base may be stearylamine, piperidine, or benzalkonium chloride and the Lewis acid may be carboxymethylcellulose, polyacrylic acid, or polyacrylic acid/polyoxyethylene copolymer, for example.

U.S. Pat. No. 4,740,365, Yukimatsu et al., issued Apr. 26, 1988 describes a sustained-release preparation applicable to mucous membranes in the oral cavity. The preparation consists of an active ingredient in a mixture of a polymer component (A) comprising one or more polymers selected from polyvinylpyrrolidone, polyvinyl alcohol, polyethylene glycol, alginic acid or a salt thereof, and an alternating copolymer of maleic anhydride and methyl vinyl ether and a polymer component (B) comprising one or more polymers selected from polyacrylic acid and a salt thereof. Polymer component (A) and (B) are in a ratio of 95:5 to 5:95 by weight. The preparation is layered with the active ingredient and may have optional conventional carriers and additives.

U.S. Pat. No. 5,451,411, Gombotz et al., issued Sep. 19, 1995, describes a delivery system for a cationic therapeutic agent whereupon alginate has been cross-linked in the presence of the therapeutic agent and polyacrylic acid to obtain a sustained release composition for oral delivery.

U.S. Pat. No. 5,352,448, Bowersock et al., issued Oct. 4, 1994, describes an oral vaccine formulation comprising an enzymatically degradable antigen in a hydrogel matrix for stimulation of an immune response in gut-associated lymphoid tissues. The hydrogel pellets are preferably synthesized by polymerizing methacrylic acid, in the presence of methylene bis-acrylamide and ammonium persulfate and sodium bisulfite.

U.S. Pat. No. 5,674,495, Bowersock et al., issued Oct. 7, 1997, describes a vaccine composition for oral administration comprising an alginate gel in the form of discrete particles. The alginate gel may contain a polymer coating such as poly-l-lysine to enhance stability and to add a positive charge to the surface.

U.S. Pat. No. 4,944,942, Brown et al., issued Jul. 31, 1990, describes an intranasal vaccine for horses, which may comprise polyacrylic acid cross linked polyallyl sucrose, sold as Carbopol 934P, combined with polyoxyethylene sorbitan mono-oleate and sorbitan monolaurate, preferably at 7.5 to 15 volume percent based on the total volume of the formulation, as an adjuvant.

U.S. Pat. No. 5,500,161, Andrianov et al., issued Mar. 19, 1996, describes a method for the preparation of microparticles, and the product thereof, that includes dispersing a substantially water insoluble non-ionic or ionic polymer in a aqueous solution in which the substance to be delivered is also dissolved, dispersed or suspended, and then coagulating the polymer together with the substance by impact forces to form a microparticle. Alternatively, the microparticle is formed by coagulation of an aqueous polymeric dispersion through the use of electrolytes, pH changes, organic solvents in low concentrations, or temperature changes to form polymer matrices encapsulating biological materials.

U.S. Pat. No. 6,015,576, See et al., issued Jan. 18, 2000, describes a method that comprises orally administering lyophilized multilamellar liposomes containing the antigen wherein the liposome preparation is contained in a pill form or within an enterically coated capsule. Such an enteric coating may be composed of acrylic polymers and copolymers.

U.S. Pat. No. 5,811,128, Tice et al., issued Sep. 22, 1998, describes a method, and compositions, for delivering a bioactive agent to an animal entailing the steps of encapsulating effective amounts of the agent in a biocompatible excipient to form microcapsules having a size less than approximately ten micrometers and administering effective amounts of the microcapsules to the animal. A pulsatile response is obtained, as well as mucosal and systemic immunity. The biocompatible excipient is selected from the group consisting of poly (DL-lactide-co-glycolide), poly (lactide), poly (glycolide), copolyoxalates, polycaprolactone, polyorthoesters and poly (beta-hydroxybutyric acid), polyanhydrides and mixtures thereof.

U.S. Pat. No. 5,565,209, Rijke, issued Oct. 15, 1996, describes oil-free vaccines comprising polyoxypropylene-polyoxyethylene polyols and an acrylic acid polymer as adjuvant constituents for injectable vaccines.

U.S. Pat. No. 5,084,269, Kullenberg, issued Jan. 28, 1992, describes an adjuvant, comprised of lecithin in combination with a carrier which may be selected from the group consisting of non-edible oil such as mineral oil and edible triglyceride oils such as soybean oil, for an injectable vaccine.

U.S. Pat. No. 5,026,543, Rijke, issued Jun. 25, 1991, discloses oil-free vaccines which contain polyoxypropylene-polyoxyethylene polyols as well as an acrylic acid polymer as adjuvanting constituents.

U.S. Pat. No. 5,451,411, Gombotz et al, issued Sep. 19, 1995, discloses alginate beads as a site specific oral delivery system for cationic therapeutic agents designed to target the agents to the luminal side of the small intestine. Enhanced bioactivity of therapeutic agents released from the alginate is attributed to the ability of polyacrylic acid to shield the agents from interaction with lower molecular weight fragments of acid treated alginate.

U.S. Pat. No. 5,716,637, Anselem et al., issued Feb. 10, 1998, describes a vaccine composition that is a solid-fat nanoemulsion that consists of a lipid core stabilized by a phospholipid membrane for the delivery of fat-soluble and water-soluble immunogens.

U.S. Pat. No. 5,567,433, Collins, issued Oct. 22, 1996, discloses a method of producing liposomes useful for encapsulating and delivering a wide variety of biologically active materials. The method involves the formation of a liposome dispersion in the absence of an organic solvent or detergent, one or several cycles of freezing and thawing, and dehydration to form a lipid powder. The powder is hydrated in the presence of a biologically active material to encapsulate it in the liposomes.

U.S. Pat. No. 5,091,188, Haynes, issued Feb. 25, 1992, discloses water-insoluble drugs that are rendered injectable by formulation as aqueous suspensions of phospholipid-coated microcrystals.

U.S. Pat. No. 6,676,958, Gerber, issued Jan. 13, 2004, describes an adjuvant for vaccines that consists of lecithin and a polymer or copolymer of acrylic acid that is useful in protecting the antigen from degradation in the stomach, adsorbing the vaccine antigen onto mucosal surfaces, and enhancing its penetration to underlying mucosal surfaces.

The chemical nature of adjuvants, their mode of action and their side effects continue to remain highly variable. Accordingly, there remains a need in the art for alternative adjuvant compositions that are highly effective, can be readily manufactured, and can be efficiently delivered to a human or animal to stimulate a desired immune response.

### SUMMARY OF THE INVENTION

In accordance with an important aspect of the invention, there is provided an adjuvant composition that includes a calcium compound, lecithin, and a polymer that preferably comprises an acrylic polymer or copolymer. In some embodiments, the preferred calcium compound comprises calcium phosphate or calcium chloride and the preferred acrylic polymer comprises a polyacrylic acid polymer.

The addition of a calcium compound to the adjuvant composition unexpectedly results in a stronger immune response to vaccine antigens than has been observed in the absence of such a calcium compound. Further, the use of a calcium compound in combination with lecithin and an acrylic polymer or copolymer mitigates adverse reactions that may otherwise occur at the site of delivery when using calcium containing compounds.

In an important application, the antigen is comprised of DNA or a killed or attenuated bacteria, virus, parasite, or structural subunits thereof. In other important applications, the adjuvant composition of the present invention can also comprise a composition such as an antibiotic, probiotic, or antiviral.

Accordingly, it is an object of the present invention to provide novel adjuvant compositions and methods of delivering the adjuvant compositions in a vaccine that can facilitate the administration of antigens to produce a stronger immune response while avoiding adverse reactions at the site of delivery.

Further features and advantages of the present invention will be set forth in, or become apparent from, the detailed description of preferred embodiments thereof which follows.

### DETAILED DESCRIPTION OF THE PREFERRED EMBODIMENT

Unless defined otherwise, all technical and scientific terms used herein have the same meaning as commonly understood to one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” or “an antigen” includes a plurality of such cells or antigens, respectively, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed compositions and methods.

The term “antigen” is used herein to refer to a substance which, when introduced into an animal or a human or expressed in a human or animal, will result in the formation of antibodies and cell-mediated immunity.

The term “adjuvant” is used herein to refer to a compound or compounds that, when used in combination with specific vaccine antigens in formulations, augment or otherwise alter or modify the resultant immune responses.

The term “vaccine” is used herein to refer to a composition of antigenic moieties, usually consisting of modified-live (attenuated) or inactivated infectious agents, or some part of the infectious agents, that is administered, most often with an adjuvant, into the body of an animal or human to produce active immunity.

One aspect of the present invention concerns an adjuvant composition which, when mixed with an antigen and administered to a human or animal, induces a stronger immune response to the antigen than when the antigen is administered alone or when the antigen is administered with an adjuvant comprising lecithin and a polymer but not containing a calcium compound. An example of the latter is the adjuvant described in U.S. Pat. No. 6,676,958, which is hereby incorporated herein by reference in its entirety. Another aspect of the present invention concerns compositions comprising an antigen or a group of antigens and an adjuvant comprised of a calcium compound, lecithin, and an acrylic polymer. A further aspect of the present invention concerns methods of using the above-mentioned compositions.

An important advantage of an adjuvant compositions in accordance with the invention is that adverse reactions are avoided at the site of delivery. Further, without wishing to be bound by any particular theory, it is believed that the addition of a calcium compound provides a cationic charge to the lipid vesicles which further facilitates delivery of the antigen to cells. Further, calcium itself is believed to play a role in the transfer of DNA across the plasma membrane of cells.

Many different antigens can be used in the adjuvant-antigen composition described above, and such antigens include those which are commercially available and those which one of ordinary skill in this art are capable of producing. The antigenic moiety making up the vaccine can be, for example, either a modified-live or killed microorganism, or a natural product purified from a microorganism or other cell including, but not limited to, tumor cell, a synthetic product, a genetically engineered protein, peptide, polysaccharide or similar product, or an allergen. The antigenic moiety can also be a subunit of a protein, peptide, polysaccharide or similar product. The antigen can also be an antigen derived from genetic materials, such as DNA or RNA that upon uptake and expression in a cell produces a polypeptide antigen. Representative of the antigens that can be used according to the present invention include, but are not limited to, natural, recombinant or synthetic products derived from viruses, bacteria, fungi, parasites and other infectious agents in addition to autoimmune diseases, hormones, or tumor antigens which might be used in prophylactic or therapeutic vaccines and allergens. The viral or bacterial products can be components which the organism produced by enzymatic cleavage or can be components of the organism that were produced by recombinant DNA techniques that are well known to those of ordinary skill in the art. Because of the nature of the invention, the invention can also function as a delivery system for substances such as hormones, antibiotics and antivirals.

The above-mentioned calcium compound preferably comprises any calcium compound that can provide a cationic charge to the lipid vesicles and/or provides a stronger immune response to the antigen. As such, in some embodiments, the calcium compound can be compounds such as calcium phosphate and calcium chloride. In some embodiments of the present invention, the calcium compound is preferably calcium phosphate.

The lecithin for use in this invention can be any lecithin or, for instance, lecithin lipoidal material, such as phosphotidylcholine, that can be used to form liposomes. Phospholipids, lysophospholipids, glycolipids and neutral lipids comprise the typical composition of lecithin. Lecithins are molecules that, when completely hydrolyzed, yield two molecules of fatty acid, and one molecule each of glycerol, phosphoric acid, and a basic nitrogenous compound, which is usually choline. The fatty acids obtained from lecithins on hydrolysis include, but are not limited to, oleic, palmitic, and stearic acids. The phosphoric acid can be attached to the glycerol in either an alpha- or beta-position, forming alpha-glycerophosphoric acid or beta-glycerophosphoric acid, respectively, and producing the corresponding series of lecithins which are known as alpha- and beta-lecithins.

Commercial lecithin is obtained by extraction processes from egg yolk, brain tissue, or soybeans. Ovolecithin (vitelin) from eggs and vegilecithin from soybeans, as well as purified lecithin from calf's brains have been used as emulsifiers, antioxidants, and stabilizers in foods and pharmaceutical preparations. Commercial lecithin can be obtained from a variety of sources, for example Central Soya (Fort Wayne, Ind.). One of ordinary skill in the art would be able to determine an appropriate lecithin for a desired application. In some embodiments, the lecithin is preferably a de-oiled lecithin, such as, for example, Solex P.

The polymer is preferably an acrylic polymer, which is any polymer or copolymer that contains an acrylic moiety. Examples of suitable acrylic polymers include, but are not limited to polyacrylic acid, methacrylic acid, methacrylate, acrylamide, acrylate, acrylnitrile, and alkyl-esters of poly acrylic acid. Examples of acrylic copolymers are poly (acrylamide-co butyl, methacrylate), acrylic-methacrylic acid, acrylic-acrylamide and poly (methacrylate). Examples of commercially available acrylic polymers include, Carbopol (Lubrizol Corp., Wickliffe, Ohio), Carboset (Lubrizol Corp., Wickliffe, Ohio), Neocryl (Avecia, Inc., Wilmington, Del.), and Eudragit (Rohm Tech, Inc., Malden, Mass.).

One method of manufacturing the above-described adjuvant first involves hydrating the lecithin and polymer by suspending from about 0.0001 to about 10% by weight/volume dry lecithin; from about 0.0001 to about 10% by weight polymer; from about 0.001 to about 10%; or, from about 0.001 to about 1.0% by weight of the calcium compound in saline or water. Preferred concentrations of lecithin and polymer, in some embodiments, are about 0.001 to about 1.0% each by weight/volume. A preferred concentration of the calcium compound is, in some embodiments, from about 0.4% to about 0.6% by weight/volume. In some embodiments, the calcium compound is calcium phosphate, which can be added to the composition at a concentration of about 0.5% by weight.

The three components may be mixed together using conventional methods, such as, for example, a Waring® blender, emulsification equipment or a microfluidizer. In an exemplary method of preparing the composition, the lecithin and polymer are mixed together with calcium subsequently added to the lecithin/polymer mixture. Various antigens and/or DNA plasmids can then be added to mixture containing lecithin, a polymer, and a calcium compound. Surfactants (emulsifiers) may be added to aid in the mixing or emulsification of the lecithin and polymer. Suitable surfactants are well known to those of ordinary skill in the art. Examples of appropriate surfactants include polyoxyethylene sorbitan monooleate, sorbitan monolaurate, sodium stearate, non-ionic ether-linked surfactants such as Laureth®4 and Laureth®23, alkyl sulfate surfactants, alkyl alkoxylated sulfate surfactants, alkylbenzenesulphonates, alkanesulphonates, olefinsulphonates, sulphonated polycarboxylic acids, alkyl glycerol sulfonates, fatty acyl glycerol sulfonates, fatty oleyl glycerol sulfates, alkyl phenol ethylene oxide ether sulfates, paraffin sulfonates, alkyl phosphates, isothionates such as the acyl isothionates, N-acyl taurates, fatty acid amides of methyl tauride, alkyl succinamates and sulfosuccinates, mono- and diesters of sulfosuccinate, N-acyl sarcosinates, sulfates of alkylpolysaccharides, branched primary alkyl sulfates, alkyl polyethoxy carboxylates, and fatty acids esterified with isethionic acid and neutralized with sodium hydroxide. Further examples are given in Surface Active Agents and Detergents (Vol. I and II by Schwartz, Perry and Berch), the disclosure of which is incorporated herein by reference. Suitable nonionic detergent surfactants are generally disclosed in U.S. Pat. No. 3,929,678, Laughlin et al., issued Dec. 30, 1975, at column 13, line 14 through column 16, line 6, incorporated herein by reference. If included, the emulsifier should be added in a concentration ranging from about 0.001-0.05% by volume of the mixture.

Although the above-described adjuvant itself, of course, has adjuvant properties, it may also be used in combination with other adjuvants including, but not limited to, saponins, fractions of saponins, synthesized components of saponins, ISCOMS, muramyl dipeptide and analogues, pluronic polyols, trehalose dimycolate, amine containing compounds, cytokines and lipopolysaccharide derivatives. The addition of another adjuvant may aid in the stimulation of a mucosal immune response. If included, additional adjuvants may be present in a concentration of up to about 10% by weight of the composition, with less than about 1% by weight being preferred. The determination and adjustment of the amount of a further adjuvant, as well as an evaluation of when and how to make such adjustments, can be made by those of ordinary skill in the art using only routine experimentation.

One or more probiotics can also be included. Probiotics are bacteria or microorganisms that are beneficial to the health of the individual or animal. Examples of commonly used probiotics include, but are not limited to, various beneficial strains of Lactobacillus, Bifidobacterium, Streptococcus, etc. If present, each of the organisms should be administered in a concentration ranging from about 103 to 108 CFU each.

In addition to all of the above, as is well understood by those skilled in the art, other minor ingredients can be employed to make the basic composition more pharmaceutically and/or cosmetically desirable. For example, dyes can be added at very minor levels as can diluents such as alcohol, buffers, stabilizers, wetting agents, dissolving agents, colors, etc. With the exception of diluents such as alcohols which are used at higher levels, the levels of these minors are generally not more than 0.001% to 1.0% by weight.

The adjuvant composition can be used for the delivery of vaccine antigens such as whole killed or attenuated virus, bacteria, or parasite vaccine antigens or sub-unit(s) of such organisms parenterally or to mucosal surfaces, such as the oral cavity, gut, nasal, vaginal and rectal surfaces. Further, the adjuvant composition can be used for the delivery of DNA into cells, wherein the DNA, such as a plasmid, is taken up into cells, where expression of a target antigen encoded by the DNA occurs. The resulting proteins and/or polypeptides can then undergo processing as intracytoplasmic antigens and produce peptides that bind to Class I MHC molecules. The presentation of these MHC bound peptides on the cell surface then stimulates an immune response. Without wishing to be bound by any particular theory, it is believed that the calcium compounds that are included in the present adjuvant composition, provide a cationic charge to the lipid vesicles that facilitates delivery of the antigen, including the delivery of DNA, to the cells.

Binding of an antigen can be further enhanced by the electrical charge and the hydrophilic and hydrophobic properties of lecithin and the acrylic polymers of this invention. To facilitate this, a polymer of different electrical charge may be selected depending on the anionic or cationic properties of the antigen. Likewise a polymer and lecithin of different hydrophobicity may be selected depending on the lipophilic or hydrophilic properties of the antigen. The selection of a different polymer, as well as an evaluation of when and how to make such a selection, can be made by those of ordinary skill in the art using only routine experimentation.

The strong mucoadhesive and adsorptive properties of the acrylic acid/lecithin combination also make the present adjuvant composition containing calcium compounds an excellent mechanism to aid in the adsorption of vaccine antigen onto mucosal surfaces. The adjuvant delivery system's absorption enhancement properties help bring the vaccine antigen in contact with mucosal associated lymphoid tissue. Thus, an immune response is engendered that will aid in the protection of an animal from infections and/or disease process. A robust mucosal immune response is critical since most infectious disease-causing organisms gain entry to the animal at mucosal surfaces.

The vaccine comprising the adjuvant composition is delivered to a mucosal surface by direct application, ingestion through the oral cavity, insertion, injection, and through other conventional means known in the art. Alternatively, the adjuvant may also be administered as a conventional injectable, which is typically either a liquid solution or suspension. When administered in an injectable, the adjuvant/vaccine composition should be present in a concentration ranging from about 0.02 to about 2.0% by weight, with about 0.1 to about 0.5% by weight being preferred. Actual dosage levels of active ingredients in a composition of the presently-disclosed subject matter can be varied so as to administer an amount of the active compound(s), such as antigens including DNA plasmids, that is effective to achieve the desired therapeutic response for a particular subject and/or application. The selected dosage level will depend upon a variety of factors including the activity of the therapeutic composition, formulation, the route of administration, combination with other drugs or treatments, the size and type of the subject being treated, and the physical condition and prior medical history of the subject being treated. Preferably, a minimal dose is administered. Determination and adjustment of a therapeutically effective dose, as well as evaluation of when and how to make such adjustments, are known to those of ordinary skill in the art of medicine.

The adjuvant/vaccine may also be administered in other conventional solid dosage forms, such as in tablets, capsules, granules, troches, and vaginal or rectal suppositories. If administered in a solid dosage form, the adjuvant/vaccine composition should constitute between about 0.0001 to about 10% by weight of the dosage form, with about 0.01 to about 1.0% by weight being preferred.

In addition to the active compounds, the above-described pharmaceutical compositions can also contain suitable excipients and auxiliaries which facilitate processing of the active compounds into preparations which can be used pharmaceutically. Oral dosage forms encompass tablets, capsules, and granules. Preparations which can be administered rectally include suppositories. Other dosage forms include suitable solutions for administration parenterally or orally, and compositions which can be administered buccally or sublingually.

The pharmaceutical preparations are preferably manufactured in a manner which is itself well known in the art. For example, the pharmaceutical preparations may be made by means of conventional mixing, granulating, dissolving, lyophilizing processes. The processes to be used will depend ultimately on the physical properties of the active ingredient used.

Suitable excipients are, in particular, fillers such as sugars for example, lactose or sucrose mannitol or sorbitol, cellulose preparations, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Oral dosage forms may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added. Possible pharmaceutical preparations which can be used rectally include, for example, suppositories, which consist of a combination of the active compounds with the suppository base. Suitable suppository bases are, for example, natural or synthetic triglycerides, paraffin hydrocarbons, polyethylene glycols, or higher alkanols. In addition, it is also possible to use gelatin rectal capsules which consist of a combination of the active compounds with a base. Possible base material include for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, including for example, sodium carboxymethyl cellulose, sorbitol and/or dextran. Such compositions may also comprise adjuvants such as preserving, wetting, emulsifying, and dispensing agents. They may also be sterilized, for example, by filtration through a bacteria-retaining filter, or by incorporating sterilizing agents into the compositions. They can also be manufactured in the form of sterile solid compositions which can be dissolved or suspended in sterile water, saline, or other injectable medium prior to administration.

In addition to administration with conventional carriers, active ingredients may be administered by a variety of specialized delivery drug techniques which are known to those of skill in the art, such as portable infusion pumps.

Use of the present composition comprised of a calcium compound, lecithin, and a polymer for the delivery of antigens including, but not limited to, antigens produced after the delivery of DNA into cells, unexpectedly results in a stronger immune response. Further, without wishing to be bound by theory, employing calcium in the present composition is believed to provide a cationic charge which further facilitates delivery of the antigen and/or DNA into cells. One problem with the use of calcium, either alone or in combination with most adjuvants, is that an adverse reaction can occur at the site of delivery. However, it has unexpectedly been found that when a calcium compound is used in the present adjuvant composition, no such adverse reaction is observed. Methods of determining whether an adverse reaction has occurred are known to those of ordinary skill in the art and include, but are not limited to, methods such as visually assessing swelling at the site of injection and histological methods such as a microscopic evaluation of tissue to determine whether immune cells including, but not limited to, mast cells and basophils, have infiltrated the area and caused an inflammatory response.

The presently-disclosed subject matter is further illustrated by the following specific but non-limiting examples. Some of the following examples are prophetic, notwithstanding the numerical values, results and/or data referred to and contained in the examples. Further, the following examples may include compilations of data that are representative of data gathered at various times during the course of development and experimentation related to the present invention.

### EXAMPLES

### Example 1

### Preparation of DNA Vaccine and Adjuvant

A DNA vaccine and adjuvant composition was made by combining 30 μg of an Avian influenza DNA plasmid containing a eukaryotic expression vector (EEV) expressing avian influenza virus protein subtype H protein strain A/Turkey/Wisconsin/68 (HA), 1.5 mg of lecithin, 1 mg of the acrylic polymer Carbopol 934P, and 0.5% by weight calcium phosphate into 0.5 ml of sterile saline.

### Example 2

### Comparison of Vaccine Adiuvants Administered by Injection

Various compositions for delivery by injection were prepared to evaluate AMPHIGEN™ and lecithin/acrylic polymer, with and without calcium phosphate, as adjuvants to Avian Influenza Virus (AIV) DNA capable of expressing the HA protein. Chicks were divided into 7 treatment groups. Group 1 was administered only a negative plasmid. Group 2 was administered AIV plasmid DNA and no adjuvant. Group 3 was administered AIV plasmid DNA plus AMPHIGEN™. Group 4 was administered AIV plasmid DNA plus AMPHIGEN™ plus calcium phosphate. Group 5 was administered AIV plasmid DNA plus a lecithin/acrylic polymer adjuvant. Group 6 was administered AIV plasmid DNA plus a lecithin/acrylic polymer with calcium phosphate. The chicks that received the AIV plasmid each received approximately 30 μg of the AIV plasmid. Each chick received two 0.5 ml dintrmuscular injections. The chicks were injected with the various compositions on day 0 and again at day 14. Sera was collected from the chicks on day 14 and on day 28, and hemaglutination titers of chicks to the HA protein were assessed. Results are shown in Table 1. Titers of less than 8 were assigned a value of 4 for purposes of calculating the geometric mean titer (GMT).

TABLE 1
Hemaglutination inhibition titers of chicks to HA protein
following vaccination with AIV plasmid DNA combined
with different adjuvants.

Geometric
Mean Titer
(GMT)

| Treatment Group | Number of Chicks | Day 14 | Day 28 |
|---|---|---|---|
| 1 | 4 | 4 | 4 |
| 2 | 9 | 16 | 87.1 |
| 3 | 9 | 6.9 | 53.8 |
| 4 | 9 | 5.9 | 29.6 |
| 5 | 9 | 8 | 69.1 |
| 6 | 8 | 32 | 215.3 |

The GMT of chicks in Group 6, that received AIV plasmid DNA plus a lecithin/acrylic polymer with calcium phosphate, was higher than in the chicks administered the AIV plasmid DNA plus AMPHIGEN™, with or without calcium phosphate, or than in chicks that received AIV plasmid DNA with a lecithin/acrylic polymer but without calcium phosphate. These results demonstrate that the combination of calcium phosphate, lecithin, and acrylic polymer was far more effective at delivering a DNA vaccine than other similar adjuvants such as a lecithin and acrylic polymer adjuvant without calcium phosphate. No welling at the site of injection was observed in the chicks that received AIV plasmid DNA plus a lecithin/acrylic polymer with calcium phosphate.

### Example 3

### Intranasal Administration

Compositions similar to those prepared in Examples 1 and 2 are prepared for delivery by the intranasal route. The first composition is comprised of a negative plasmid alone. The second composition is comprised of only an AIV plasmid expressing the HA protein (AIV-HA plasmid). The third composition is comprised of an AIV-HA plasmid adjuvanted with calcium phosphate. The fourth composition is comprised of an AIV-HA plasmid adjuvanted with lecithin and an acrylic polymer. The fifth composition is comprised an AIV-HA plasmid adjuvanted with lecithin and acrylic polymer that further contains calcium phosphate. Chicks are vaccinated on days 0 and 20 by placing the compositions on the nostrils of the chicks while the mouth is held shut. The compositions then enter the nose of each chick when the chick inhales. On day 30, the chicks are euthanized and bled by cutting the brachial artery. The hemaglutination titers of the chicks to the HA protein are then assessed. The highest GMT is observed in chicks administered an AIV-HA plasmid adjuvanted with a lecithin and acrylic polymer that further contained calcium phosphate, thus indicating that the combination of calcium phosphate, lecithin, and acrylic polymer is far more effective at delivering a DNA vaccine to a mucosal surface than other similar adjuvants such as a lecithin and acrylic polymer adjuvant without calcium phosphate.

Throughout this document, various references are mentioned. All such references are incorporated herein by reference.

Although the invention has been described above in relation to preferred embodiments thereof, it will be understood by those skilled in the art that variations and modifications can be effected in these preferred embodiments without departing from the scope and spirit of the invention.

What is claimed is:

1. An adjuvant composition comprising a calcium compound, lecithin, and a polymer selected from the group consisting of an acrylic polymer, an acrylic copolymer, or a combination thereof.

2. The composition of claim 1, wherein the polymer is an acrylic polymer that is selected from the group consisting of polyacrylic acid, methacrylic acid, methacrylate, arylamide, acrylate, acrylnitrile, poly (acrylimide-co butyl, methacrylate), acrylic-methacrylic acid, acrylic-acrylamide, poly (methacrylate) and alkyl-esters of poly acrylic acid.

3. The composition of claim 1, wherein the lecithin and the polymer are each present in the composition at a concentration of about 0.001 to about 10% by weight.

4. The composition of claim 3, wherein the lecithin and the polymer are each present in the composition at a concentration of about 0.001 to about 1.0% by weight.

5. The composition of claim 1, wherein the calcium compound is selected from the group consisting of calcium phosphate and calcium chloride.

6. The composition of claim 1, wherein the calcium compound is present in the composition at a concentration of about 0.001 to about 10% by weight.

7. The composition of claim 6, wherein the calcium compound is present in the composition at a concentration of about 0.001 to about 1.0% by weight.

8. The composition of claim 7, wherein the calcium compound is present in the composition at a concentration of about 0.4% to about 0.6% by weight.

9. The composition of claim 1, further including a second adjuvant.

10. The composition of claim 9, wherein the second adjuvant is selected from the group consisting of saponins, fractions of saponins, synthesized fractions of saponins, ISCOMS, pluronic polyols, muramyl dipeptide and analogues thereof, trehalose dimyoclate, lipopolysaccharide derivatives, and cytokines.

11. The composition of claim 1, wherein the composition is lyophilized.

12. The composition of claim 1, further comprising a therapeutic composition selected from the group consisting of antibiotics, probiotics, and antivirals.

13. The composition of claim 1, further comprising an antigen.

14. The composition of claim 13, wherein the antigen comprises DNA or a killed or attenuated bacteria, virus, parasite, or structural subunit thereof.

15. The composition of claim 13, wherein the composition is administered in an injectable formulation.

16. A method for stimulating an immune response in a human or animal subject comprising: formulating a composition comprising an antigen and an adjuvant comprising a calcium compound, lecithin, and an acrylic polymer; and administering the composition to a human or animal subject.

17. The method of claim 16, wherein the composition is administered to a mucosal surface selected from the group consisting of oral cavity, gut, nasal, vaginal, and rectal mucosal surfaces.

18. The method of claim 16, wherein the composition is administered in an injectable formulation.

19. A pharmaceutical composition comprising an antigen and an adjuvant, wherein the adjuvant comprises a calcium compound, lecithin, and a polymer selected from the group consisting of an acrylic polymer, an acrylic copolymer, or a combination thereof.

20. The composition of claim 19, wherein the antigen comprises DNA or a killed or attenuated bacteria, virus, parasite, or structural subunit thereof.
